(19) United States
(12) Patent Application Publication
(10) Pub. No.: US 2011/0028503 A1
(43) Pub. Date: Feb. 3, 2011

(54) COMPOSITIONS AND METHODS FOR INHIBITION OF THE JAK PATHWAY

(75) Inventors: Vanessa Taylor, San Francisco, CA (US); Hui Li, Santa Clara, CA (US); Rajinder Singh, Belmont, CA (US)

Correspondence Address: Weaver Austin Villeneuve & Sampson LLP, P.O. BOX 70250, OAKLAND, CA, 94612-0250, US

(73) Assignee: RIGEL PHARMACEUTICALS, INC., South San Francisco, CA (US)

(21) Appl. No.: 12/845,655

(22) Filed: Jul. 28, 2010

Related U.S. Application Data
(60) Provisional application No. 61/229,191, filed on Jul. 28, 2009.

(51) Int. Cl. A61K 31/505; A61P 27/02; A61P 27/06; A61P 9/10; C07D 239/48; A61P 27/14

(52) U.S. Cl. 514/275; 544/327

(57) ABSTRACT

Compounds I and II as well as salts and pharmaceutical compositions containing them are useful for treating diseases and/or disorders of the eye.

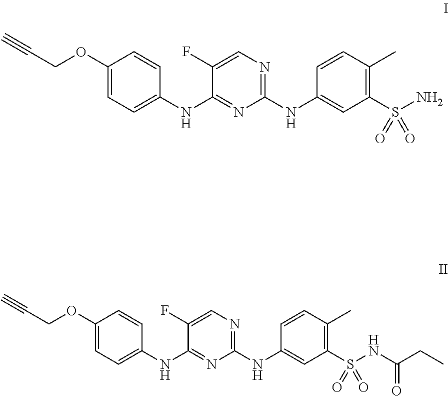

### CROSS-REFERENCE TO RELATED APPLICATIONS

This application claims the benefit of and priority to U.S. Provisional Application Ser. No. 61/229,191 filed Jul. 28, 2009, the contents of which are incorporated herein by reference in their entirety and for all purposes.

### FIELD

The present disclosure relates to compounds, prodrugs, salts thereof, and pharmaceutical compositions containing them, and methods of using these compounds, prodrugs and compositions thereof in the treatment of diseases and/or disorders of the eye.

### BACKGROUND

JAK kinases (JAnus Kinases) are a family of cytoplasmic protein tyrosine kinases including JAK1, JAK2, JAK3 and TYK2. Each of the JAK kinases is selective for the receptors of certain cytokines, though multiple JAK kinases may be affected by particular cytokine or signaling pathways. Studies suggest that JAK3 associates with the common gamma (γc) chain of the various cytokine receptors. JAK3 in particular selectively binds to receptors and is part of the cytokine signaling pathway for IL-2, IL-4, IL-7, IL-9, IL-15 and IL-21. JAK1 interacts with, among others, the receptors for cytokines IL-2, IL-4, IL-7, IL-9 and IL-21, while JAK2 interacts with, among others, the receptors for IL-9 and TNF-α. Upon binding of certain cytokines to their receptors (for example, IL-2, IL-4, IL-7, IL-9, IL-15 and IL-21), receptor oligomerization occurs, resulting in the cytoplasmic tails of associated JAK kinases being brought into proximity and facilitating the trans-phosphorylation of tyrosine residues on the JAK kinase. This trans-phosphorylation results in the activation of the JAK kinase.

Phosphorylated JAK kinases bind various STAT (Signal Transducer and Activator of Transcription) proteins. STAT proteins, which are DNA binding proteins activated by phosphorylation of tyrosine residues, function both as signaling molecules and transcription factors and ultimately bind to specific DNA sequences present in the promoters of cytokine-responsive genes. JAK/STAT signaling has been implicated in the mediation of many abnormal immune responses such as allergies, asthma, autoimmune diseases such as transplant (allograft) rejection, rheumatoid arthritis, amyotrophic lateral sclerosis and multiple sclerosis, ocular disorders and diseases, as well as in solid and hematologic malignancies such as leukemia and lymphomas.

Dry eye syndromes (keratitis sicca) represent a particularly widespread clinical problem. Dry eye disorders are generally caused by either evaporative dysfunction or aqueous tear-deficiency. The evaporative dysfunctions occur in the presence of normal lacrimal function, but may be caused by meibomian gland dysfunction, loss of normal eyelid function, or ocular surface causes (such as contact lens use or ocular allergy). Aqueous deficiencies are generally caused by either Sjogren's syndrome or non-Sjogren's disorders. Despite the varying causes of dry eye, the ultimate pathologic mechanism is breakdown of the pre-ocular tear film with dehydration of the exposed corneal surface which results in discomfort and irritation of the cornea. Upon clinical examination, signs of a dry eye may include bulbar conjunctival vascular dilation, conjunctival pleating, a decreased tear meniscus, an irregular corneal surface, and increased debris in the tear film. Diffuse corneal staining with bengal rose or fluorescein stain is usually observed, and filaments or mucous plaques may be seen in more advanced cases.

A variety of clinical tests have been used for the diagnosis of dry eyes. These tests include inspection of the depth of the tear meniscus, decreased tear breakup time, and the Schirmer test.

A common cause of dry eyes is Sjogren's syndrome, which is an autoimmune disorder in which immune cells attack and impair the glands that produce tears and saliva. The hallmark symptoms of the disorder are dry mouth and dry eyes. Sjögren's syndrome affects 1-4 million people in the United States, with women being nine times more likely to develop the disease. Sjögren's syndrome can occur as a primary condition or as a secondary disorder in association with other autoimmune diseases, such as systemic lupus erythematosus (“lupus”) or rheumatoid arthritis. Dry eyes are frequently seen in association with other common disorders, such as rosacea. Dry eyes are also a common side-effect of many medications, such as isotretinoin, diuretics, tricyclic antidepressants, sedatives, antihypertensive medications, oral contraceptives, antihistamines, nasal decongestants, and many others.

In spite of the prevalence of dry eye conditions, the primary treatment is still the use of artificial tears (such as carboxymethyl cellulose, hydroxy methycellulose, or hydroxypropyl cellulose) to moisten the cornea and provide temporary symptomatic relief. Another common treatment has been punctual occlusion, in which the outflow of tears from the eye can be partially interrupted by occluding the ducts which normally serve to drain them. Treatment of underlying disorders can also improve the symptoms of dry eye, for example by treating rosacea with an appropriate antibiotic. It would nonetheless be desirable to provide a treatment that provides relief for a variety of dry eye disorders, such as (but not limited to) immune-mediated keratitis sicca.

### SUMMARY

In view of the numerous conditions that may benefit by treatment involving modulation of the JAK pathway, it has now been appreciated that compounds that modulate JAK pathways and methods of using these compounds should provide substantial therapeutic benefit to a wide variety of patients.

Disclosed are compounds, prodrugs, corresponding salt forms, and methods of using these compounds, prodrugs and salt forms in the treatment of diseases and/or disorders of the eye.

One embodiment provides a compound I, and solvates, prodrugs and pharmaceutically acceptable salts thereof:

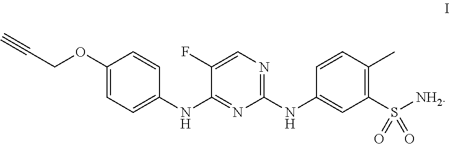

One embodiment provides a particular prodrug of compound I, and pharmaceutically acceptable salt forms thereof, which is compound II:

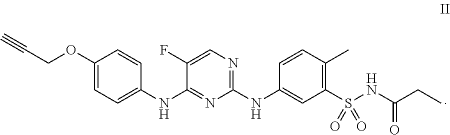

In one aspect, ocular disorders are treated using an effective amount compound I and/or II, as well as salt forms thereof and pharmaceutical compositions which include the compound or compounds. One embodiment provides a method of treating a disease and/or disorder of the eye, comprising administering to a subject an amount of compound I and/or II effective to treat the disease and/or disorder of the eye. Diseases and disorders of the eye treated using the presently disclosed compounds include, but are not limited to, dry eye syndrome, diabetic retinopathy, macular degeneration (such as age-related macular degeneration), uveitis, allergic conjunctivitis, glaucoma and rosacea. In particular examples, the disorder is a dry eye disorder, such as keratitis or keratoconjunctivitis sicca, for example a disorder caused by deficient tear production or abnormal tear composition, such as a disorder of the tear glands. In particular non-limiting examples, the disorder is an autoimmune or immune mediated disorder, such as Sjogren's syndrome. In other examples, the disorder is idiopathic keratitis sicca or rosacea.

In one aspect of the disclosed method for treating ocular disorders, administration of one or more of the presently disclosed 2,4-pyrimidinediamine compounds is effective to increase tear production volume as compared to untreated tear production volume, thereby ameliorating a symptom of dry eye syndrome. In one aspect, tear production volume is increased within five days, such as in less than four days, and in some examples in less than two days. In one embodiment, tear production volume is increased by at least about 25% over initial tear production within two days of initial treatment with a presently disclosed 2,4-pyrimidinediamine compound. In other embodiments, tear production is increased at least about 30%, such as at least about 50% over initial tear production within less than two days. Increases in tear production upon administration of the present compounds results, in some instances, in tear production volume comparable to normal tear production.

In another aspect, the compound of formula I and/or II, or the pharmaceutically acceptable salt form thereof, is administered either in combination or adjunctively with an anti-inflammatory, an antihistamine, an antibiotic, an antiviral and a glaucoma medication. In particular examples, the anti-inflammatory agent may be a non-steroidal anti-inflammatory agent (NSAID) or corticosteroid (such as prednisolone) or immunosuppressant (such as cyclosporine A) administered either systemically (for example orally or parenterally) or topically (in eye drops). A monoclonal antibody (such as cytokine blocker) or an agent that inhibits an miR gene product could also be used. In other examples, the treatment is combined with non-pharmaceutical treatments, such as punctal occlusion or fitting the subject with scleral or semi-scleral contact lenses that create a fluid-filled layer over the cornea. Yet other combination treatments can include concomitant or adjunctive treatment of an underlying disorder associated with the dry eyes, for example treatment of roseacea with an anti-rosacea drug or regimen such as a tetracycline class antibiotic (for example minocycline or doxycycline), or laser surgery to reduce facial erythema or rhinophyma.

Typically the disclosed compounds of formula I and/or II, when used for treating ocular disorders topically, typically are administered at least once daily, such as twice daily.

In another embodiment, this invention provides a pharmaceutical formulation comprising compound I and/or II, either in parent or salt form, and at least one pharmaceutically acceptable excipient, diluent, preservative, or stabilizer, or mixtures thereof. In another example, the pharmaceutical formulation is a combination formulation that also includes a non-steroidal or steroidal anti-inflammatory agent or other treatment for dry eyes, including combination formulations that are intended to treat underlying conditions (such as Sjogren's disease or rosacea) that are contributing to the keratitis sicca. In yet other examples, the compound I and/or II is administered in a lubricant composition, particularly a viscous composition, that provides a vehicle for topical ocular administration of the drug as well as symptomatic relief from the dry eyes.

These and other embodiments are described in more detail below.

### DETAILED DESCRIPTION

### Definitions

As used herein, the following definitions shall apply unless otherwise indicated.

“Corticosteroids” are steroid hormones that are produced in the adrenal cortex. Corticosteroids are involved in a wide range of physiologic systems such as stress response, immune response and regulation of inflammation, carbohydrate metabolism, protein catabolism, blood electrolyte levels, and behavior. Examples of corticosteroids include cortisol, prednisone and prednisilone. Corticosteroids can be administered either orally, parenterally (for example by injection) or by direct topical instillation in the eye with eye drops, and they may be combined with the compounds of formula I and/or II in a combination formulation.

“Keratitis sicca” or “keratoconjunctivitis sicca” or “dry eye syndrome” refers to a dry eye condition associated with decreased tear production or increased evaporation, and having various etiologies, such as inflammatory, non-inflammatory, traumatic, iatrogenic, drug-induced, rosacea-associated, or idiopathic origins. A particular cause of dry eyes is decreased production of tears from the tear glands, for example because of an autoimmune associated condition that impairs the proper function of the lacrimal glands or meibomian glands. The condition can be treated either with drugs or non-drug interventions, such as punctal occlusion (for example by introducing a plug into the puncta or using electrocauterization of ablate or partially ablate the punctal opening or tear duct).

“Non-steroidal anti-inflammatory drug (NSAID)” is a type of anti-inflammatory agent that works by inhibiting the production of prostaglandins. NSAIDS exert anti-inflammatory, analgesic and antipyretic actions. Examples of NSAIDS include ibuprofen, ketoprofen, piroxicam, naproxen, sulindac, aspirin, choline subsalicylate, diflunisal, fenoprofen, indomethacin, meclofenamate, salsalate, tolmetin and magnesium salicylate. These agents can be administered either orally, parenterally (for example by injection) or by direct topical instillation in the eye with eye drops, and they may be combined with the compounds of formula I and/or II in a combination formulation.

“Subject” refers to humans and non-human subjects.

“Pharmaceutically acceptable salt” refers to pharmaceutically acceptable salts of a compound, which salts are derived from a variety of organic and inorganic counter ions well known in the art.

“Pharmaceutically effective amount” or “therapeutically effective amount” refers to an amount of a compound sufficient to treat a specified disorder or disease or one or more of its symptoms and/or to prevent the occurrence of the disease or disorder.

The phrase “restoration of normal tear production” refers to the cessation of dry eye symptoms as described in standard ophthalmic practice, such as a response score of less than 14.5 in McMonnies & Ho Dry Eye questionnaire, test results (for example red phenol, fluorescein and the like as is known to those skilled in ophthamological practice) or a combination of indications.

As used herein, the phrase “significantly increases tear production” means a statistically significant (such as p<0.05) increase in tear production as measured by standard ophthalmic practice. For example tear production can be measured by the Schirmer test, the phenol red thread test, tear breakup time (such as by fluorescein staining), Rose Bengal staining, and the like.

### Compounds

Disclosed are compounds, prodrugs, corresponding salt forms, and methods of using these compounds, prodrugs and salt forms in the treatment of diseases and/or disorders of the eye.

Compounds I and II, as well as their salt forms and pharmaceutical compositions containing them are described in more detail below. Compound I is also referred to as N2-(3-aminosulfonyl-4-methylphenyl)-5-fluoro-N4-[4-(prop-2-ynyloxy)phenyl]-2,4-pyrimidinediamine. Compound II is also referred to as 5-fluoro-N2-(4-methyl-3-propionylaminosulfonylphenyl)-N4-[4-(prop-2-ynyloxy)phenyl]-2,4-pyrimidinediamine.

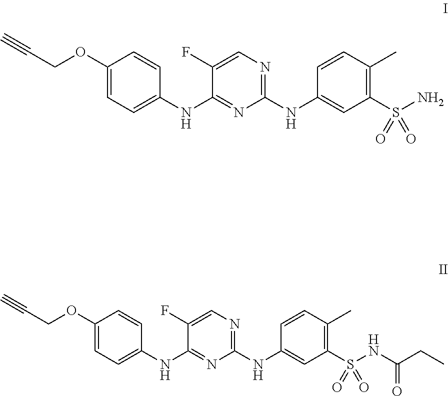

For the purposes of brevity in description, for any embodiment where compound I and compound II are referred to specifically, there is a corresponding embodiment where a salt form and/or a pharmaceutical composition containing compound I and/or compound II are used.

One of ordinary skill in the art will appreciate that compound II is a prodrug of compound I, and that compound II need not necessarily be, pharmacologically inactive until converted into compound I. The mechanism by which the propionyl progroup metabolizes is not critical, and can be caused by, for example, hydrolysis under the acidic conditions of the stomach, and/or by enzymes present in the digestive tract and/or tissues or organs of the body, for example, esterases, amidases, lipolases, phosphatases including ATPases and kinases, cytochrome P450's of the liver, and the like. In particular embodiments described herein, compounds I and/or II are used to treat ocular disorders and may therefore be administered directly to the eye. In some embodiments, administration may include not only topical but also injection and the like. These modes of administration do not preclude, for example, therapeutic benefit to the eye via systemic circulation of compound I and/or II.

One of ordinary skill in the art will appreciate that compounds I and II, may exhibit the phenomena of tautomerism, conformational isomerism and/or geometric isomerism. It should be understood that the invention encompasses any tautomeric, conformational isomeric and/or geometric isomeric forms of the compounds as well as mixtures of these various different isomeric forms. Atropisomers are stereoisomers resulting from hindered rotation about single bonds where the barrier to rotation is high enough to allow for the isolation of the conformers (Eliel, E. L.; Wilen, S. H. Stereochemistry of Organic Compounds; Wiley & Sons: New York, 1994; Chapter 14). Atropisomerism is significant because it introduces an element of chirality in the absence of stereogenic atoms. The invention is meant to encompass atropisomers, for example in cases of limited rotation about bonds between the 2,4-pyrimidinediamine core structure and groups attached thereto or for example about bonds between the sulfonamide and the phenyl ring to which it is attached. Compounds I and II may be in the form of salts. Such salts include salts suitable for pharmaceutical uses (“pharmaceutically-acceptable salts”), salts suitable for veterinary uses, etc. Such salts may be derived from acids or bases, as is well-known in the art. Exemplary salts described herein are sodium salts, potassium salts, arginine salts, choline salts and calcium salts, but generically any pharmaceutically acceptable salt may be used for methods described herein. Because compound I and compound II have both basic groups, for example pyrimidine nitrogens, and acidic groups, for example acidic protons on the sulfonamide and/or the nitrogens at N2 and N4 of the pyrimidinediamine system, these compounds can form pharmaceutically acceptable acid or base addition salts.

In one embodiment, the salt is a pharmaceutically acceptable salt. Generally, pharmaceutically acceptable salts are those salts that retain substantially one or more of the desired pharmacological activities of the parent compound and which are suitable for administration to humans. Pharmaceutically acceptable salts include acid addition salts formed with inorganic acids or organic acids. Inorganic acids suitable for forming pharmaceutically acceptable acid addition salts include, by way of example and not limitation, hydrohalide acids (for example, hydrochloric acid, hydrobromic acid, hydroiodic acid, etc.), sulfuric acid, nitric acid, phosphoric acid, and the like. Organic acids suitable for forming pharmaceutically acceptable acid addition salts include, by way of example and not limitation, acetic acid, trifluoroacetic acid, propionic acid, hexanoic acid, cyclopentanepropionic acid, glycolic acid, oxalic acid, pyruvic acid, lactic acid, malonic acid, succinic acid, malic acid, maleic acid, fumaric acid, tartaric acid, citric acid, palmitic acid, benzoic acid, 3-(4-hydroxybenzoyl)benzoic acid, cinnamic acid, mandelic acid, alkylsulfonic acids (for example, methanesulfonic acid, ethanesulfonic acid, 1,2-ethane-disulfonic acid, 2-hydroxyethanesulfonic acid, etc.), arylsulfonic acids (for example, benzenesulfonic acid, 4-chlorobenzenesulfonic acid, 2-naphthalenesulfonic acid, 4-toluenesulfonic acid, camphorsulfonic acid, etc.), 4-methylbicyclo[2.2.2]-oct-2-ene-1-carboxylic acid, glucoheptonic acid, 3-phenylpropionic acid, trimethylacetic acid, tertiary butylacetic acid, lauryl sulfuric acid, gluconic acid, glutamic acid, hydroxynaphthoic acid, salicylic acid, stearic acid, muconic acid, and the like.

Pharmaceutically acceptable salts also include salts formed when an acidic proton present in the parent compound is either replaced by a metal ion (for example, an alkali metal ion, an alkaline earth metal ion or an aluminum ion) or coordinates with an organic base (for example, ethanolamine, diethanolamine, triethanolamine, N-methylglucamine, morpholine, piperidine, dimethylamine, diethylamine, triethylamine, ammonia, etc.).

Compounds I and II, as well as the salts thereof, may also be in the form of solvates, for example hydrates, and N-oxides, as are well-known in the art.

### Methods

The present invention provides 2,4-substituted pyrimidinediamine compounds I and II, prodrugs, salts and pharmaceutical compositions thereof, for use in treating diseases and/or disorders of the eye. In particular, compounds I and II, alone or in combination with other agents. As described, compound I and/or compound II, can be administered as the parent and/or the salt form, and as pharmaceutical formulations thereof.

As used herein, and as well understood in the art, “treatment” is an approach for obtaining beneficial or desired results, including clinical results. For the purposes of this invention, beneficial or desired results can include one or more, but are not limited to, alleviation or amelioration of one or more symptoms, diminishment of extent of a condition, including a disease, stabilized (i.e., not worsening) state of a condition, including diseases, preventing spread of disease, delay or slowing of condition, including disease, progression, amelioration or palliation of the condition, including disease, state, and remission (whether partial or total), whether detectable or undetectable. Compounds I and II (at least as a source of compound I) are potent, and thus can be administered locally at very low doses, thus minimizing systemic adverse effects.

Compounds I and II are potent and selective inhibitors of JAK kinases, and particularly selective for cytokine signaling pathways containing JAK3. As a consequence of this activity, the compounds may be used in a variety of in vitro, in vivo and ex vivo contexts to regulate or inhibit JAK kinase activity, signaling cascades in which JAK kinases play a role, and the biological responses effected by such signaling cascades. For example, in one embodiment, the compounds may be used to inhibit JAK kinase, either in vitro or in vivo, in virtually any cell type expressing the JAK kinase. For example, in hematopoietic cells, in which, for example JAK3 is predominantly expressed. They may also be used to regulate signal transduction cascades in which JAK kinases, particularly JAK3, play a role. Such JAK-dependent signal transduction cascades include, but are not limited to, the signaling cascades of cytokine receptors that involve the common gamma chain, such as, for example, the IL-4, IL-7, IL-5, IL-9, IL-15 and IL-21, or IL-2, IL-4, IL-7, IL-9, IL-15 and IL-21 receptor signaling cascades. The compounds may also be used in vitro or in vivo to regulate, and in particular inhibit, cellular or biological responses affected by such JAK-dependent signal transduction cascades. Such cellular or biological responses include, but are not limited to, IL-4/ramos CD23 upregulation, IL-2 mediated T-cell proliferation, etc. Importantly, the compounds may be used to inhibit JAK kinases in vivo as a therapeutic approach towards the treatment or prevention of diseases mediated, either wholly or in part, by a JAK kinase activity. Such diseases are referred to as “JAK kinase mediated diseases.”

While not wishing to be bound by theory, it is believed that compounds described herein are effective treatments of these eye disorders due, at least in part, to their JAK inhibitory activity. Examples of diseases that are mediated, at least in part, by JAK kinases that can be treated or prevented according to the methods include diseases and disorders of the eye including, but not limited to, dry eye syndrome, uveitis, allergic conjunctivitus, glaucoma, sympathetic ophthalmia and rosacea (of the eye). However, as a result of the aforementioned activities, although methods described herein are directed to treatment of ocular disorders, administration of the compounds and/or formulations may carry other therapeutic benefit, that is, in other tissues or organs of the body. One embodiment is a method of treating an ocular disorder or disease, where a secondary benefit is also realized. As mentioned, one embodiment provides a method of treating a disease and/or disorder of the eye, comprising administering to a subject an amount of a compound effective to treat the disease and/or disorder of the eye wherein the compound is selected from compound I and compound II. Diseases and disorders of the eye include, but are not limited to, dry eye syndrome, uveitis, allergic conjunctivitus, glaucoma and rosacea (of the eye). Dry eye syndrome (DES), otherwise known as keratoconjunctivitis sicca (KCS), keratitis sicca, sicca syndrome, or xerophthalmia, is an eye disease caused by decreased tear production or increased tear film evaporation commonly found in humans and some animals. Uveitis or iridocyclitis refers to inflammation of the middle layer of the eye (the “uvea”) and in common usage may refer to any inflammatory process involving the interior of the eye. Allergic conjunctivitis is inflammation of the conjunctiva (the membrane covering the white part of the eye) due to allergy. Glaucoma refers to a group of diseases that affect the optic nerve and involves a loss of retinal ganglion cells in a characteristic pattern, i.e., a type of optic neuropathy. Raised intraocular pressure is a significant risk factor for developing glaucoma (above 22 mmHg or 2.9 kPa), and inflammatory processes, e.g uveitis, can cause this rise in intraocular pressure.

Rosacea is a chronic inflammatory condition characterized by facial erythema but it can affect the eyes and nose (rhinophyma). The present methods include treating both topical facial erythema, rhinophyma and ocular rosacea. In one embodiment, the disease and/or disorder of the eye is selected from dry eye syndrome, diabetic retinopathy, macular degeneration uveitis, allergic conjunctivitus, glaucoma rosacea and combinations thereof. In one embodiment, the disease and/or disorder of the eye is dry eye syndrome. In another embodiment, the disease and/or disorder of the eye is uveitis. In one embodiment, the disease and/or disorder of the eye is allergic conjunctivitus. In one embodiment, disease and/or disorder of the eye is glaucoma. In another embodiment, the disease and/or disorder of the eye is rosacea.

In one embodiment, compound I and/or compound II are used to treat any of the aformentioned ocular diseases and/or disorders. In one embodiment, compound I and/or II are employed as salt forms. In a particular embodiment, compound II is used as a salt form. In one embodiment, the salt of compound II is selected from the sodium salt, the potassium salt, the calcium salt, the arginine salt and the choline salt.

### Co-Administration

When used to treat eye diseases, compounds I and II may be administered singly, as mixtures and/or in combination with other agents useful for treating diseases and/or disorders of the eye. Compounds I and II may be administered in mixture or in combination with agents, useful to treat other disorders or maladies, such as steroids, membrane stabilizers, 5-lipoxygenase (5LO) inhibitors, leukotriene synthesis and receptor inhibitors, inhibitors of IgE isotype switching or IgE synthesis, IgG isotype switching or IgG synthesis, β-agonists, tryptase inhibitors, aspirin, cyclooxygenase (COX) inhibitors, methotrexate, anti-TNF drugs, rituxan, PD4 inhibitors, p38 inhibitors, PDE4 inhibitors, and antihistamines, to name a few. Compounds I and II may be administered per se, in the form of prodrugs, or as pharmaceutical compositions, comprising the active compound and/or prodrug.

Particular immunosuppressive therapies that can be used in combination with compounds I and II include, for example, mercaptopurine, corticosteroids such as prednisone, methylprednisolone and prednisolone, alkylating agents such as cyclophosphamide, calcineurin inhibitors such as cyclosporine, sirolimus and tacrolimus, inhibitors of inosine monophosphate dehydrogenase (IMPDH) such as mycophenolate, mycophenolate mofetil and azathioprine, and agents designed to suppress cellular immunity while leaving the recipient's humoral immunologic response intact, including various antibodies (for example, antilymphocyte globulin (ALG), antithymocyte globulin (ATG), monoclonal anti-T-cell antibodies (OKT3)) and irradiation. These various agents can be used in accordance with their standard or common dosages, as specified in the prescribing information accompanying commercially available forms of the drugs (see also, the prescribing information in the 2006 Edition of The Physician's Desk Reference), the disclosures of which are incorporated herein by reference. Azathioprine is currently available from Salix Pharmaceuticals, Inc. under the brand name AZASAN; mercaptopurine is currently available from Gate Pharmaceuticals, Inc. under the brand name PURINETHOL; prednisone and prednisolone are currently available from Roxane Laboratories, Inc.; Methyl prednisolone is currently available from Pfizer; sirolimus (rapamycin) is currently available from Wyeth-Ayerst under the brand name RAPAMUNE; tacrolimus is currently available from Fujisawa under the brand name PROGRAF; cyclosporine is current available from Novartis under the brand dame SANDIMMUNE and Abbott under the brand name GENGRAF; IMPDH inhibitors such as mycophenolate mofetil and mycophenolic acid are currently available from Roche under the brand name CELLCEPT and Novartis under the brand name MYFORTIC; azathioprine is currently available from Glaxo Smith Kline under the brand name IMURAN; and antibodies are currently available from Ortho Biotech under the brand name ORTHOCLONE, Novartis under the brand name SIMULECT (basiliximab) and Roche under the brand name ZENAPAX (daclizumab).

In one embodiment, the compound of formula I and/or II, or the pharmaceutically acceptable salt form thereof, is administered either in combination or adjunctively with an antihistamine, an antibiotic, an anti-inflammatory, an antiviral and a glaucoma medication. Examples of common antibiotics used in the eye are sulfacetamide, erythromycin, gentamicin, tobramycin, ciprofloxacin and ofloxacin. Corticosteroids (sometimes referred to as “steroids”) are similar to a natural substance produced by the adrenal gland and are very effective anti-inflammatories for a wide variety of eye problems. Corticosteroids can be safely used in the eye, and do not carry most of the risks associated with oral steroids like prednisone. Corticosteroids used to treat the eye include, but are not limited to, prednisolone, fluorometholone and dexamethasone. Non-steroidal anti-inflammatories for the eye include, but are not limited to, ibuprofen, diclofenac, ketorolac and flurbiprofen. Common antihistamines include livostin, patanol, cromolyn, alomide. There are also non-prescription antihistamines for the eye, which are less potent but can be very helpful in milder case, such as pheniramine. Common antiviral eye medications include, but are not limited to, triflurthymidine, adenine, arabinoside and idoxuridine. Glaucoma medications typically attempt to reduce the eye's intraocular pressure, the fluid pressure inside the eye, to prevent damage to the optic nerve resulting in loss of vision. These medications may lower pressure by decreasing the amount of fluid produced in the eye, by increasing the amount of fluid exiting through the eye's natural drain, or by providing additional pathways for fluid to leave the eye. Often more than one glaucoma medication will be used simultaneously, as these effects can combine to lower pressure even further than possible with one medicine alone. Common glaucoma medications include, but are not limited to, betablockers such as timolol, metipranolol, carteolol, betaxolol and levobunolol; prostaglandin analogues such as latanoprost; cholinergic agonists such as pilocarpine and carbachol; alpha agonists such as bromonidine and iopidine; carbonic anhydrase inhibitors such as dorzolamide; and adenergic agonists such as epinephrine and dipivefrin.

### Pharmaceutical Compositions

Pharmaceutical compositions comprising compounds I and II described herein can be manufactured by means of conventional mixing, dissolving, granulating, dragee-making levigating, emulsifying, encapsulating, entrapping or lyophilization processes. The compositions can be formulated in a conventional manner using one or more physiologically acceptable carriers, diluents, excipients or auxiliaries which facilitate processing of the active compounds into preparations which can be used pharmaceutically.

Compounds I and II can be formulated in the pharmaceutical compositions per se, or in the form of a hydrate, solvate, N-oxide or pharmaceutically acceptable salt, as described herein. Typically, such salts are more soluble in aqueous solutions than the corresponding free acids and bases, but salts having lower solubility than the corresponding free acids and bases may also be formed.

In one embodiment, is provided a pharmaceutical formulation comprising compound I and/or compound II, and at least one pharmaceutically acceptable excipient, diluent, preservative, or stabilizer, or mixtures thereof.

In one embodiment, the compounds are provided as non-toxic pharmaceutically acceptable salts as noted previously. Suitable pharmaceutically acceptable salts of the compounds of this invention include acid addition salts such as those formed with hydrochloric acid, fumaric acid, p-toluenesulphonic acid, maleic acid, succinic acid, acetic acid, citric acid, tartaric acid, carbonic acid or phosphoric acid. Salts of amine groups may also comprise quaternary ammonium salts in which the amino nitrogen atom carries a suitable organic group such as an alkyl, alkenyl, alkynyl or aralkyl moiety. Furthermore, where the compounds described herein carry an acidic moiety, suitable pharmaceutically acceptable salts thereof may include metal salts such as alkali metal salts, for example sodium or potassium salts; and alkaline earth metal salts, for example calcium or magnesium salts.

The pharmaceutically acceptable salts described herein may be formed by conventional means, such as by reacting the free base form of the product with one or more equivalents of the appropriate acid in a solvent or medium in which the salt is insoluble, or in a solvent such as water which is removed in vacuo or by freeze drying or by exchanging the anions of an existing salt for another anion on a suitable ion exchange resin.

Compounds I and II may be administered by oral, parenteral (for example, intramuscular, intraperitoneal, intravenous, ICV, intracisternal injection or infusion, subcutaneous injection, or implant), by inhalation spray, nasal, vaginal, rectal, sublingual, urethral (for example, urethral suppository) or topical routes of administration (for example, gel, ointment, cream, aerosol, etc.) and may be formulated, alone or together, in suitable dosage unit formulations containing conventional non-toxic pharmaceutically acceptable carriers, adjuvants, excipients and vehicles appropriate for each route of administration. In addition to the treatment of warm-blooded animals such as mice, rats, horses, cattle, sheep, dogs, cats, monkeys, etc., the compounds described herein may be effective in humans.

The pharmaceutical compositions for the administration of compounds I and II may conveniently be presented in dosage unit form and may be prepared by any of the methods well known in the art of pharmacy. The pharmaceutical compositions can be, for example, prepared by uniformly and intimately bringing the active ingredient into association with a liquid carrier or a finely divided solid carrier or both, and then, if necessary, shaping the product into the desired formulation. In the pharmaceutical composition the active object compound is included in an amount sufficient to produce the desired therapeutic effect. For example, pharmaceutical compositions described herein may take a form suitable for virtually any mode of administration, including, for example, topical, ocular, oral, buccal, systemic, nasal, injection, transdermal, rectal, vaginal, etc., or a form suitable for administration by inhalation or insufflation.

For topical administration, the JAK-selective compound(s) or prodrug(s) may be formulated as solutions, gels, ointments, creams, suspensions, etc. as are well-known in the art. In particular, solutions, gels, ointments, creams and suspensions are well-suited for administration directly to the eye. One embodiment is a pharmaceutical formulation comprising compound I and/or compound II, where the formulation is selected from a solution, a gel, an ointment, a cream and a suspension. In one embodiment, the formulation is a solution. In another embodiment, the formulation is a gel. In another embodiment, the formulation is a suspension. In yet another embodiment, the formulation is a cream or ointment. One embodiment is any of the aforementioned formulations in a kit for administration to the eye, either topically or via injection into the eye. In one embodiment, the formulation is a liquid, for example a homogeneous liquid or a suspension, sold in a bottle which dispenses the formulation as eye drops. In one embodiment, the formulation is a cream or ointment, sold in a tube which dispenses the formulation to the eye, for example, under the eye lid. In another embodiment, the compound is provided in a viscous liquid (such as carboxylmethylcellulose, hydroxypropylmethycellulose, polyethylene glycol, glycerin, polyvinyl alcohol, or oil containing drops) for instillation in the eye. The formulations may have preservatives or be preservative-free (for example in a single-use container). The use of preservative-free preparations can be of particular benefit in the treatment of dry eyes, which are often sensitive to and made worse by the introduction of preservatives in the eye.

Systemic formulations include those designed for administration by injection, for example, subcutaneous, intravenous, intramuscular, intrathecal or intraperitoneal injection, as well as those designed for transdermal, transmucosal oral or pulmonary administration.

Useful injectable preparations include sterile suspensions, solutions or emulsions of the active compound(s) in aqueous or oily vehicles. The compositions may also contain formulating agents, such as suspending, stabilizing and/or dispersing agent. The formulations for injection may be presented in unit dosage form, for example, in ampules or in multidose containers, and may contain added preservatives.

Alternatively, the injectable formulation may be provided in powder form for reconstitution with a suitable vehicle, including but not limited to sterile pyrogen free water, buffer, dextrose solution, etc., before use. To this end, the active compound(s) may be dried by any art-known technique, such as lyophilization, and reconstituted prior to use.

For transmucosal administration, penetrants appropriate to the barrier to be permeated are used in the formulation. Such penetrants are known in the art.

For oral administration, the pharmaceutical compositions may take the form of, for example, lozenges, tablets or capsules prepared by conventional means with pharmaceutically acceptable excipients such as binding agents (for example, pregelatinised maize starch, polyvinylpyrrolidone or hydroxypropyl methylcellulose); fillers (for example, lactose, microcrystalline cellulose or calcium hydrogen phosphate); lubricants (for example, magnesium stearate, talc or silica); disintegrants (for example, potato starch or sodium starch glycolate); or wetting agents (for example, sodium lauryl sulfate). The tablets may be coated by methods well known in the art with, for example, sugars, films or enteric coatings. Additionally, the pharmaceutical compositions containing the 2,4-substituted pyrmidinediamine as active ingredient or prodrug thereof in a form suitable for oral use, may also include, for example, troches, lozenges, aqueous or oily suspensions, dispersible powders or granules, emulsions, hard or soft capsules, or syrups or elixirs. Compositions intended for oral use may be prepared according to any method known to the art for the manufacture of pharmaceutical compositions and such compositions may contain one or more agents selected from the group consisting of sweetening agents, flavoring agents, coloring agents and preserving agents in order to provide pharmaceutically elegant and palatable preparations. Tablets contain the active ingredient (including prodrug) in admixture with non-toxic pharmaceutically acceptable excipients which are suitable for the manufacture of tablets. These excipients may be for example, inert diluents, such as calcium carbonate, sodium carbonate, lactose, calcium phosphate or sodium phosphate; granulating and disintegrating agents (for example, corn starch, or alginic acid); binding agents (for example starch, gelatin or acacia); and lubricating agents (for example magnesium stearate, stearic acid or talc). The tablets may be uncoated or they may be coated by known techniques to delay disintegration and absorption in the gastrointestinal tract and thereby provide a sustained action over a longer period. For example, a time delay material such as glyceryl monostearate or glyceryl distearate may be employed. They may also be coated by the techniques described in the U.S. Pat. Nos. 4,256,108; 4,166,452; and 4,265,874 to form osmotic therapeutic tablets for control release. The pharmaceutical compositions described herein may also be in the form of oil-in-water emulsions.

Liquid preparations for oral administration may take the form of, for example, elixirs, solutions, syrups or suspensions, or they may be presented as a dry product for constitution with water or other suitable vehicle before use. Such liquid preparations may be prepared by conventional means with pharmaceutically acceptable additives such as suspending agents (for example, sorbitol syrup, cellulose derivatives or hydrogenated edible fats); emulsifying agents (for example, lecithin or acacia); non-aqueous vehicles (for example, almond oil, oily esters, ethyl alcohol, Cremophore™ or fractionated vegetable oils); and preservatives (for example, methyl or propyl-p-hydroxybenzoates or sorbic acid). The preparations may also contain buffer salts, preservatives, flavoring, coloring and sweetening agents as appropriate.

Preparations for oral administration may be suitably formulated to give controlled release of the active compound or prodrug, as is well known.

For buccal administration, the compositions may take the form of tablets or lozenges formulated in conventional manner.

For rectal and vaginal routes of administration, the active compound(s) may be formulated as solutions (for retention enemas) suppositories or ointments containing conventional suppository bases such as cocoa butter or other glycerides.

For nasal administration or administration by inhalation or insufflation, the active compound(s) or prodrug(s) can be conveniently delivered in the form of an aerosol spray from pressurized packs or a nebulizer with the use of a suitable propellant, for example, dichlorodifluoromethane, trichlorofluoromethane, dichlorotetrafluoroethane, fluorocarbons, carbon dioxide or other suitable gas. In the case of a pressurized aerosol, the dosage unit may be determined by providing a valve to deliver a metered amount. Capsules and cartridges for use in an inhaler or insufflator (for example capsules and cartridges comprised of gelatin) may be formulated containing a powder mix of the compound and a suitable powder base such as lactose or starch.

The pharmaceutical compositions may be in the form of a sterile injectable aqueous or oleagenous suspension. This suspension may be formulated according to the known art using those suitable dispersing or wetting agents and suspending agents which have been mentioned above. The sterile injectable preparation may also be a sterile injectable solution or suspension in a non-toxic parenterally-acceptable diluent or solvent. Among the acceptable vehicles and solvents that may be employed are water, Ringer's solution and isotonic sodium chloride solution. Compounds I and II may also be administered in the form of suppositories for rectal or urethral administration of the drug. In particular embodiments, the compounds may be formulated as urethral suppositories, for example, for use in the treatment of fertility conditions, particularly in males, for example, for the treatment of testicular dysfunction.

According to the invention, 2,4-substituted pyrimidinediamine compounds can be used for manufacturing a composition or medicament, including medicaments suitable for rectal or urethral administration. The invention also relates to methods for manufacturing compositions including 2,4-substituted pyrimidinediamine compounds in a form that is suitable for urethral or rectal administration, including suppositories.

For topical use, creams, ointments, jellies, gels, solutions or suspensions, etc., containing compounds I and II may be employed. Compounds I and II can be used for manufacturing a composition or medicament, including medicaments suitable for topical administration. In certain embodiments, compounds I and II may be formulated for topical administration with polyethylene glycol (PEG). These formulations may optionally comprise additional pharmaceutically acceptable ingredients such as diluents, stabilizers and/or adjuvants. In particular embodiments, the topical formulations are formulated for the treatment of ocular diseases and/or disorders.

Included among the devices which may be used to administer particular examples of compounds I and II are those well-known in the art, such as, metered dose inhalers, liquid nebulizers, dry powder inhalers, sprayers, thermal vaporizers, and the like. Other suitable technology for administration of particular 2,4-substituted pyrimidinediamine compounds includes electrohydrodynamic aerosolizers. Sprays and aerosols can be used to administer the compounds, either per se or in formulations, directly to the eye.

A variety of vehicles suitable for administering compounds I and II to the eye are known in the art. Specific non-limiting examples are described in U.S. Pat. No. 6,261,547; U.S. Pat. No. 6,197,934; U.S. Pat. No. 6,056,950; U.S. Pat. No. 5,800,807; U.S. Pat. No. 5,776,445; U.S. Pat. No. 5,698,219; U.S. Pat. No. 5,521,222; U.S. Pat. No. 5,403,841; U.S. Pat. No. 5,077,033; U.S. Pat. No. 4,882,150; and U.S. Pat. No. 4,738,851. Typically formulations for ocular administration contain a pharmaceutically effective amount of a 2,4-pyrimidinediamine compound disclosed herein, such as from about 0.0001% to about 1.0% by weight (w/w). In certain formulations, the pharmaceutically effective amount of the compound is 0.0003% to about 0.1% (w/w), such as from about 0.003% to about 0.5% (w/w), or from about 0.01% to about 0.03% (w/w).

In certain examples an ophthalmic composition containing a presently disclosed 2,4-pyrimidinediamine compound for ocular administration includes a tonicity agent, a buffer, or both. In certain examples of ophthalmic compositions the tonicity agent is a simple carbohydrate or a sugar alcohol. As is known to one of ordinary skill in the art, tonicity agents may be used in the present compositions to adjust the tonicity of the composition, preferably to that of normal tears. Examples of suitable tonicity agents include, without limitation sodium chloride, potassium chloride, magnesium chloride, calcium chloride, carbohydrates, such as dextrose, fructose, galactose, polyols, such as sugar alcohols, including by way of example, mannitol, sorbitol, xylitol, lactitol, isomalt, maltitol and combinations thereof. Compositions containing a buffer contain, in some examples, a phosphate, citrate, or both.

In one aspect, compositions for ocular administration of the presently disclosed 2,4-pyrimidinediamine compounds optionally contain a surfactant, a stabilizing polymer, or both. Surfactants are employed in certain compositions to facilitate the delivery of higher concentrations of the 2,4-pyrimidinediamine compound being administered. Such surfactants can work to solubilize the compound. Exemplary surfactants include polysorbate, poloxamer, polyoxyl 40 stearate, polyoxyl castor oil, tyloxapol, triton and sorbitan monolaurate. In certain embodiments the surfactant is selected from TritonX114, tyloxapol and combinations thereof. In still another embodiment of compositions for ocular administration, the stabilizing polymer is carbomer 974p.

The 2,4-substituted pyrimidinediamine compound(s) or prodrug(s) described herein, or compositions thereof, will generally be used in an amount effective to achieve the intended result, for example in an amount effective to treat or prevent the particular condition being treated. The compound(s) may be administered therapeutically to achieve therapeutic benefit or prophylactically to achieve prophylactic benefit. By therapeutic benefit is meant eradication or amelioration of the underlying disorder being treated and/or eradication or amelioration of one or more of the symptoms associated with the underlying disorder such that the patient reports an improvement in feeling or condition, notwithstanding that the patient may still be afflicted with the underlying disorder. For example, administration of a compound to a patient suffering from an ocular disorder due to an allergic reaction provides therapeutic benefit not only when the underlying allergic response is eradicated or ameliorated, but also when the patient reports a decrease in the severity or duration of the symptoms associated with the allergy following exposure to the allergen. Therapeutic benefit also includes halting or slowing the progression of the disease, regardless of whether improvement in symptoms is realized.

For prophylactic administration, the compound may be administered to a patient at risk of developing one of the previously described conditions. For example, if it is unknown whether a patient is allergic to a particular drug, the compound may be administered prior to administration of the drug to avoid or ameliorate an allergic response to the drug. Alternatively, prophylactic administration may be applied to avoid the onset of symptoms in a patient diagnosed with the underlying disorder. For example, a compound may be administered to an allergy sufferer prior to expected exposure to the allergen. Compounds may also be administered prophylactically to healthy individuals who are repeatedly exposed to agents known to one of the above-described maladies to prevent the onset of the disorder. For example, a compound may be administered to a healthy individual who is repeatedly exposed to an allergen known to induce allergic reaction in the eyes, such as pollen, in an effort to prevent the individual from developing an allergy.

The amount of compound administered will depend upon a variety of factors, including, for example, the particular condition being treated, the mode of administration, the severity of the condition being treated and the age and weight of the patient, the bioavailability of the particular active compound, etc. Determination of an effective dosage is well within the capabilities of those skilled in the art. A skilled practitioner will be able to determine the optimal dose for a particular individual. Effective dosages may be estimated initially from in vitro assays. For example, an initial dosage for use in animals may be formulated to achieve a circulating blood or serum concentration of active compound that is at or above an IC₅₀ of the particular compound as measured in as in vitro assay. Calculating dosages to achieve such circulating blood or serum concentrations taking into account the bioavailability of the particular compound is well within the capabilities of skilled artisans.

For guidance, the reader is referred to Fingl & Woodbury, “General Principles,” In: Goodman and Gilman's The Pharmaceutical Basis of Therapeutics, Chapter 1, pp. 1-46, latest edition, Pergamon Press, and the references cited therein.

Initial dosages can also be estimated from in vivo data, such as animal models. Animal models useful for testing the efficacy of compounds to treat or prevent the various diseases described above are well-known in the art. Dosage amounts will typically be in the range of from about 0.0001 or 0.001 or 0.01 mg/kg/day to about 100 mg/kg/day, but may be higher or lower, depending upon, among other factors, the activity of the compound, its bioavailability, the mode of administration and various factors discussed above. Dosage amount and interval may be adjusted individually to provide plasma levels of the compound(s) which are sufficient to maintain therapeutic or prophylactic effect. For example, the compounds may be administered once per week, several times per week (for example, every other day), once per day or multiple times per day, depending upon, among other things, the mode of administration, the specific indication being treated and the judgment of the prescribing physician. In cases of local administration or selective uptake, such as local topical administration, the effective local concentration of active compound(s) may not be related to plasma concentration. Skilled artisans will be able to optimize effective local dosages without undue experimentation.

The foregoing disclosure pertaining to the dosage requirements for the 2,4-substituted pyrimidinediamine compounds is pertinent to dosages required for prodrugs, with the realization, apparent to the skilled artisan, that the amount of prodrug(s) administered will also depend upon a variety of factors, including, for example, the bioavailability of the particular prodrug(s) the conversation rate and efficiency into active drug compound under the selected route of administration, etc. Determination of an effective dosage of prodrug(s) for a particular use and mode of administration is well within the capabilities of those skilled in the art.

Effective dosages may be estimated initially from in vitro activity and metabolism assays. For example, an initial dosage of prodrug for use in animals may be formulated to achieve a circulating blood or serum concentration of the metabolite active compound that is at or above an IC₅₀ of the particular compound as measured in as in vitro assay, such as the in vitro CHMC or BMMC and other in vitro assays described in U.S. application Ser. No. 10/355,543 filed Jan. 31, 2003 (US2004/0029902A1), international application Serial No. PCT/US03/03022 filed Jan. 31, 2003 (WO 03/063794), U.S. application Ser. No. 10/631,029 filed Jul. 29, 2003, international application Serial No. PCT/US03/24087 (WO2004/014382), U.S. application Ser. No. 10/903,263 filed Jul. 30, 2004, and international application Serial No. PCT/US2004/24716 (WO005/016893). Calculating dosages to achieve such circulating blood or serum concentrations taking into account the bioavailability of the particular prodrug via the desired route of administration is well within the capabilities of skilled artisans. For guidance, the reader is referred to Fingl & Woodbury, “General Principles,” In: Goodman and Gilman's The Pharmaceutical Basis of Therapeutics, Chapter 1, pp. 1-46, latest edition, Pergamon Press, and the references cited therein. For ocular administration, effective dosages may be those where no significant systemic circulation of the compounds results from administration to the eye, for example, where eye drops are added to the eye to treat an ocular disorder and a very localized dose is utilized prior to significant systemic circulation.

Additional compounds that can be substituted for compounds I and II in the disclosed methods are specifically contemplated herein and are described in Argade et al. U.S. Pat. No. 7,491,732, issued Feb. 17, 2009 and US Patent Application Publication No. 2007/0203161, published Aug. 30, 2007, both of which are incorporated herein by reference.

### Synthesis of the Compounds

Compounds I and II, as well as salts III-VII, are synthesized as described below or by analogy to the syntheses described below. Alternative syntheses would be appreciated by one of ordinary skill in the art.

### Example 1

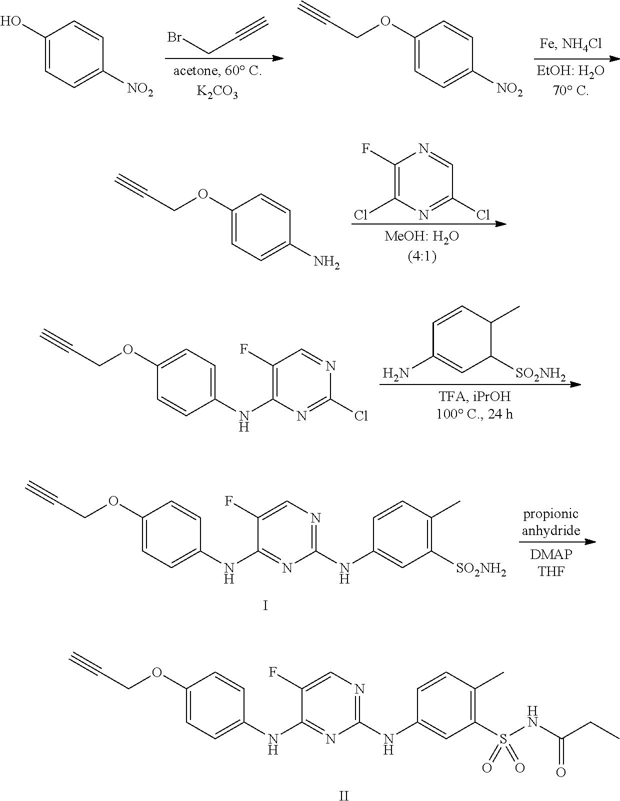

### I: N2-(3-Aminosulfonyl-4-methylphenyl)-5-fluoro-N4-[4-(prop-2-ynyloxy)phenyl]-2,4-pyrimidinediamine

4-Nitrophenol (1.00 g, 7.19 mmol), propargyl bromide (80 wt % in toluene; 0.788 mL, 7.09 mmol), and K₂CO₃ (1.08 g, 7.84 mmol) were combined and stirred in acetone (16.0 mL) at 60° C. for 18 h. The reaction mixture was cooled to room temperature and diluted with water (200 mL). 4-(prop-2-ynyloxy)nitrobenzene was isolated as a white solid by suction filtration (1.12 g). ¹H NMR (CDCl₃): δ 8.22 (d, J=9.0 Hz, 2H), 7.05 (d, J=9.0 Hz, 2H), 4.80 (d, J=2.4 Hz, 2H), 2.59 (t, J=2.4 Hz, 1H).

4-(Prop-2-ynyloxy)nitrobenzene (0.910 g, 5.13 mmol), iron (1.42 g, 25.3 mmol), and NH₄Cl (0.719 g, 12.8 mmol) were vigorously stirred in EtOH/water (1:1, 55 mL) at 70° C. for 15 minutes. The reaction mixture was filtered hot through diatomaceous earth and concentrated in vacuo. The residue was suspended in 10% 2N ammoniacal methanol in dichloromethane, sonicated, and filtered through diatomaceous earth. Concentration gave 4-(prop-2-ynyloxy)aniline as an oil which was used without further purification. ¹H NMR (CDCl₃): δ 6.82 (d, J=8.7 Hz, 2H), 6.64 (d, J=8.7 Hz, 2H), 4.61 (d, J=2.4 Hz, 2H), 2.50 (t, J=2.4 Hz, 1H).

4-(prop-2-ynyloxy)aniline (0.750 g, 5.10 mmol) and 2,4-dichloro-5-fluoropyrimidine (1.27 g, 0.760 mmol, commercially available from Sigma-Aldrich of Milwaukee, Wis., USA) were stirred in MeOH/water (4:1, 35 mL) at room temperature for 18 h. The reaction mixture was diluted with EtOAc (200 mL) and washed with 1N HCl (50 mL) and brine (50 mL). The organic layer was dried (MgSO₄), filtered and concentrated in vacuo. The residue was purified by column chromatography (silica gel, hexanes ramped to EtOAc:hexanes (1:10)) to provide 2-chloro-5-fluoro-N4-[4-(prop-2-ynyloxy)phenyl]-4-pyrimidineamine as a light brown solid (0.514 g). ¹H NMR (CDCl₃): δ 8.03 (d, J=2.7 Hz, 1H), 7.53 (d, J=8.7 Hz, 2H), 7.02 (d, J=8.7 Hz, 2H), 6.86 (s, 1H), 4.71 (d, J=2.4 Hz, 2H), 2.55 (t, J=2.4 Hz, 1H); LCMS: purity: 99%; MS (m/e): 279 (MH⁺).

2-Chloro-5-fluoro-N4-[4-(prop-2-ynyloxy)phenyl]-4-pyrimidineamine (0.514 g, 1.85 mmol), 3-(aminosulfonyl)-4-methylaniline (0.689 g, 3.70 mmol, made by reduction of commercially available 2-methyl-5-nitrobenzenesulfonamide or synthesized as described below), and trifluoroacetic acid (0.186 mL, 2.41 mmol) were combined with iPrOH (6.0 mL) in a sealed vial and heated at 100° C. for 3 h. The reaction mixture was cooled to room temperature and diluted with 1N HCl (80 mL). N2-(3-Aminosulfonyl-4-methylphenyl)-5-fluoro-N4-[4-(prop-2-ynyloxy)phenyl]-2,4-pyrimidinediamine (I) was isolated as a white solid by suction filtration (0.703 g). ¹H NMR (DMSO-d₆): δ 10.08 (bs, 2H), 8.19 (d, J=4.5 Hz, 1H), 7.89 (s, 1H), 7.74 (dd, J=2.4 and 8.4 Hz, 1H), 7.58 (d, J=8.7 Hz, 2H), 7.32 (bs, 2H), 7.23 (d, J=8.4 Hz, 1H), 6.97 (d, J=8.4 Hz, 2H), 4.79 (d, J=2.1 Hz, 2H), 3.59-3.55 (m, 1H), 2.53 (s, 3H); LCMS: purity: 97%; MS (m/e): 428 (MH⁺).

### II: 5-fluoro-N2-(4-methyl-3-propionylaminosulfonylphenyl)-N4-[4-(prop-2-ynyloxy)phenyl]-2,4-pyrimidinediamine

N2-(3-Aminosulfonyl-4-methylphenyl)-5-fluoro-N4-[4-(prop-2-ynyloxy)phenyl]-2,4-pyrimidinediamine, I, (0.200 g, 0.467 mmol), DMAP (40 mg, 0.33 mmol)) and triethylamine (0.118 mL, 0.847 mmol) were stirred in THF (6.0 mL). Propionic anhydride (0.180 mL, 1.40 mmol) was added to the solution drop wise. The reaction mixture was stirred at room temperature overnight. The solution was diluted with ethyl acetate (50 mL) and washed with water (5×25 mL) and brine (10 mL). The organic layer was dried (MgSO₄), filtered, and evaporated. The residue was suspended in ethyl acetate (25 mL), sonicated and the solid collected by filtration to give 5-fluoro-N2-(4-methyl-3-propionylaminosulfonylphenyl)-N4-[4-(prop-2-ynyloxy)phenyl]-2,4-pyrimidinediamine, II, (0.20 g). ¹H NMR (DMSO-d₆): δ 12.01 (s, 1H), 9.44 (s, 1H), 9.26 (s, 1H), 8.16 (d, J=2.4 Hz, 1H), 8.06 (dd, J=0.3 and 3.3 Hz, 1H), 8.00 (dd, J=2.1 and 7.8 Hz, 1H), 7.69 (d, J=8.7 Hz, 2H), 7.19 (d, J=8.4 Hz, 1H), 6.95 (d, J=8.7 Hz, 2H), 4.77 (d, J=2.1 Hz, 2H), 3.56 (t, J=2.1 Hz, 1H), 2.49 (s, 3H), 2.24 (q, J=7.2 Hz, 2H), 0.89 (t, J=7.2 Hz, 3H); LCMS: purity: 98%; MS (m/e): 484 (MH⁺).

### III: 5-fluoro-N2-(4-methyl-3-propionylaminosulfonylphenyl)-N4-[4-(prop-2-ynyloxy)phenyl]-2,4-pyrimidinediamine mono-sodium salt

5-Fluoro-N2-(4-methyl-3-propionylaminosulfonylphenyl)-N4-[4-(prop-2-ynyloxy)phenyl]-2,4-pyrimidinediamine, II, (0.125 g, 0.258 mmol) was suspended in acetonitrile (1.5 mL) and water (1.5 mL) and cooled in an ice bath. A solution of 1N NaOH aq. (0.260 mL) was added drop wise. The reaction mixture was stirred until it became clear, filtered through glass wool, and lyophilized to give the sodium salt of II. ¹H NMR (DMSO-d₆): δ 9.17 (bs, 2H), 8.01 (d, J=3.6 Hz, 1H), 7.89 (s, 1H), 7.78-7.69 (m, 3H), 6.99-6.92 (m, 3H), 4.76 (d, J=2.1 Hz, 1H), 2.43 (s, 3H), 1.95 (q, J=7.2 Hz, 2H), 0.86 (t, J=7.2 Hz, 3H); LCMS: purity: 98%; MS (m/e): 484 (MH⁺).

The following compounds were made in a similar fashion to those above.

### IV: 5-Fluoro-N2-[4-methyl-3-(N-propionylaminosulfonyl)phenyl]-N4-[4-(2-propynyloxy)phenyl]-2,4-pyrimidinediamine Potassium Salt

¹H NMR (DMSO-d₆): δ 9.16 (s, 1H), 9.14 (s, 1H), 8.01 (d, J=3.6 Hz, 1H), 7.85 (d, J=2.1 Hz, 1H), 7.75-7.70 (m, 3H), 6.97-6.92 (m, 3H), 4.76 (d, J=1.8 Hz, 2H), 3.55 (t, J=2.4 Hz, 1H), 2.42 (s, 3H), 1.91 (q, J=7.5 Hz, 2H), 0.85 (t, J=7.5 Hz, 3H); LCMS: purity: 97%; MS (m/z): 484 (parent, MH⁺).

### V: 5-Fluoro-N2-[4-methyl-3-(N-propionylaminosulfonyl)phenyl]-N4-[4-(2-propynyloxy)phenyl]-2,4-pyrimidinediamine Calcium Salt

¹H NMR (DMSO-d₆): δ 9.16 (s, 2H), 8.00 (d, J=3.6 Hz, 1H), 7.88 (d, J=1.8 Hz, 1H), 7.75-7.69 (m, 3H), 6.97-6.92 (m, 3H), 4.76 (d, J=1.8 Hz, 2H), 3.55 (t, J=2.1 Hz, 1H), 2.43 (s, 3H), 1.94 (q, J=7.5 Hz, 2H), 0.87 (t, J=7.5 Hz, 3H); LCMS: purity: 98%; MS (m/z): 484 (parent, MH⁺).

### VI: 5-Fluoro-N2-[4-methyl-3-(N-propionylaminosulfonyl)phenyl]-N4-[4-(2-propynyloxy)phenyl]-2,4-pyrimidinediamine Arginine Salt

¹H NMR (D₂O): δ 7.61 (d, J=3.9 Hz, 1H), 7.57-7.55 (m, 1H), 7.36-7.31 (m, 1H), 7.12 (d, J=8.7 Hz, 2H), 6.88 (d, J=8.7 Hz, 1H), 6.72 (d, J=9.0 Hz, 2H), 4.77-4.75 (m, 2H), 3.60 (t, J=6.0 Hz, 1H), 3.09 (t, J=6.9 Hz, 2H), 2.84-2.81 (m, 1H), 2.35 (s, 3H), 2.03 (q, J=5.7 Hz, 2H), 1.80-1.72 (m, 2H), 1.61-1.48 (m, 2H), 0.855 (t, J=7.5 Hz, 3H); LCMS: purity: 98%; MS (m/z): 484 (parent, MH⁺).

### VII: 5-Fluoro-N2-[4-methyl-3-(N-propionylaminosulfonyl)phenyl]-N4-[4-(2-propynyloxy)phenyl]-2,4-pyrimidinediamine Choline Salt

¹H NMR (DMSO-d₆): δ 9.16 (s, 2H), 8.00 (d, J=3.6 Hz, 1H), 7.85 (d, J=1.8 Hz, 1H), 7.75-7.69 (m, 3H), 6.97-6.90 (m, 3H), 5.27 (t, J=4.8 Hz, 1H), 4.76 (d, J=1.8 Hz, 2H), 3.86-3.77 (m, 2H), 3.56-3.54 (m, 1H), 3.40-3.54 (m, 2H), 3.08 (s, 9H), 2.42 (s, 3H); LCMS: purity: 99%; MS (m/z): 484 (parent, MH⁺).

### Example 2

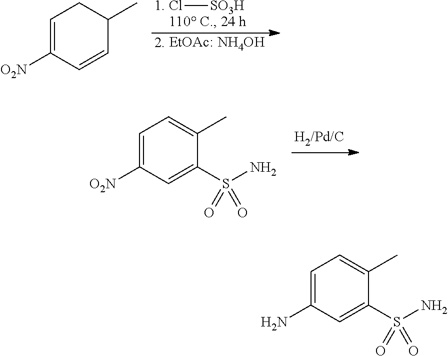

### 5-amino-2-methylbenzenesulfonamide

4-methylnitrobenzene (20 mmol) is treated at 0° C. with chlorosulfonic acid (5.29 mL, 80 mmol) and then, after bringing the homogeneous solution to room temperature, it was stirred at 110° C. for 24 hours. The resulting slurry was then poured over ice water (100 gm), extracted with diethyl ether (3×75 mL), and the organic phase washed with water (75 mL), then dried over anhydrous sodium sulfate. The solvent was then removed under reduced pressure to afford the crude sulfonyl chloride which was taken up in ethyl acetate and stirred with ammonium hydroxide overnight at room temperature. After the ethyl acetate layer was separated, the aqueous layer was extracted with ethyl acetate. The organic layers were combined, dried over anhydrous sodium sulfate and the solvent was removed under reduced pressure. The oil obtained was purified by column chromatography (silica gel, hexanes then 10%, 20%, up to 50% ethyl acetate in hexanes to afford 3-aminosulfonyl-4-methylnitrobenzene, LCMS: purity: 95%; MS (m/e): 217 (MH⁺).

To a solution of 3-aminosulfonyl-4-methylnitrobenzene in dichloromethane and methanol was added 10% Pd/C and the mixture shaken under a hydrogen atmosphere at 50 psi for 15 minutes. The mixture was filtered through diatomaceous earth and the filter cake was washed with methanol. The combined organic solvents were concentrated under reduced pressure to give crude product, which was further purified by flash column chromatography (ethyl acetate:hexanes 1:1) to give 3-aminosulfonyl-4-methylaniline, LCMS: purity: 87%; MS (m/e): 187 (MH⁺).

### Example 3

### Assay for Ramos B-Cell Line Stimulated with IL-4

One means of assaying for JAK inhibition is detection of the effect of compounds I and II on the upregulation of downstream gene products. In the Ramos/IL4 assay, B-cells are stimulated with the cytokine Interleukin-4 (IL-4) leading to the activation of the JAK/Stat pathway through phosphorylation of the JAK family kinases, JAK1 and JAK3, which in turn phosphorylate and activate the transcription factor Stat-6. One of the genes upregulated by activated Stat-6 is the low affinity IgE receptor, CD23. To study the effect of inhibitors (for example, the 2,4-substituted pyrimidinediamine compounds described herein) on the JAK1 and JAK3 kinases, human Ramos B cells are stimulated with human IL-4. Twenty to 24 hours post stimulation, cells are stained for upregulation of CD23 and analyzed using flow cytometry (FACS). A reduction of the amount of CD23 present compared to control conditions indicates the test compound actively inhibits the JAK kinase pathway. An exemplary assay of this type is described in greater detail below.

B-cells stimulated with cytokine Interleukin-4 (IL-4) activate the JAK/Stat pathway through phosphorylation of the JAK family kinases, JAK-1 and JAK-3, which in turn phosphorylate and activate the transcription factor Stat-6. One of the genes upregulated by activated Stat-6 is the low affinity IgE receptor, CD23. To study the effect of inhibitors on the JAK family kinases, human Ramos B cells are stimulated with human IL-4.

The Ramos B-cell line was acquired from ATCC (ATCC Catalog No. CRL-1596). The cells were cultured in RPMI 1640 (Cellgro, MediaTech, Inc., Herndon, Va., Cat No. 10-040-CM) with 10% fetal bovine serum (FBS), heat inactivated (JRH Biosciences, Inc, Lenexa, Kans., Cat No. 12106-500M) according to ATCC propagation protocol. Cells were maintained at a density of 3.5×10⁵. The day before the experiment, Ramos B-cells were diluted to 3.5×10⁵ cells/mL to ensure that they were in a logarithmic growth phase.

Cells were spun down and suspended in RPMI with 5% serum. 5×10⁴ cells were used per point in a 96-well tissue culture plate. Cells were pre-incubated with compound or DMSO (Sigma-Aldrich, St. Louis, Mo., Cat No. D2650) vehicle control for 1 hour in a 37° C. incubator. Cells were then stimulated with IL-4 (Peprotech Inc., Rocky Hill, N.J., Cat No. 200-04) for a final concentration of 50 units/mL for 20-24 hours. Cells were then spun down and stained with anti-CD23-PE(BD Pharmingen, San Diego, Calif., Cat No. 555711) and analyzed by FACS. Detection was performed using a BD LSR I System Flow Cytometer, purchased from Becton Dickinson Biosciences of San Jose, Calif. The IC₅₀ calculated based on the results of this assay are provided in Table 1.

### Example 4

### Primary Human T-Cell Proliferation Assay Stimulated with IL-2

The JAK activity of the compounds described herein may further be characterized by assaying the effect of compounds I and II described herein on the proliferative response of primary human T-cells. In this assay, primary human T-cells derived from peripheral blood and pre-activated through stimulation of the T-cell receptor and CD28, proliferate in culture in response to the cytokine Interleukin-2 (IL-2). This proliferative response is dependent on the activation of JAK1 and JAK3 tyrosine kinases, which phosphorylate and activate the transcription factor Stat-5. The primary human T-cells are incubated with compounds I and II in the presence of IL-2 for 72 hours and at the assay endpoint intracellular ATP concentrations are measured to assess cell viability. A reduction in cell proliferation compared to control conditions is indicative of inhibition of the JAK kinase pathway. An exemplary assay of this type is described in greater detail below.

Primary human T-cells derived from peripheral blood and pre-activated through stimulation of the T-cell receptor and CD28, proliferate in vitro in response to the cytokine Interleukin-2 (IL-2). This proliferative response is dependent on the activation of JAK-1 and JAK-3 tyrosine kinases, which phosphorylate and activate the transcription factor Stat-5.

Human primary T cells were prepared as follows. Whole blood was obtained from a healthy volunteer, mixed 1:1 with PBS, layered on to Ficoll Hypaque (Amersham Pharmacia Biotech, Piscataway, N.J., Catalog #17-1440-03) in 2:1 blood/PBS:ficoll ratio and centrifuged for 30 min at 4° C. at 1750 rpm. The lymphocytes at the serum: ficoll interface were recovered and washed twice with 5 volumes of PBS. The cells were resuspended in Yssel's medium (Gemini Bio-products, Woodland, Calif., Catalog #400-103) containing 40 U/mL recombinant IL2 (R and D Systems, Minneapolis, Minn., Catalog #202-IL (20 μg)) and seeded into a flask pre-coated with 1 μg/mL anti-CD3 (BD Pharmingen, San Diego, Calif., Catalog #555336) and 5 μg/mL anti-CD28 (Immunotech, Beckman Coulter of Brea Calif., Catalog #IM1376). The primary T-cells were stimulated for 3-4 days, then transferred to a fresh flask and maintained in RPMI with 10% FBS and 40 U/mL IL-2.

Primary T-cells were washed twice with PBS to remove the IL-2 and resuspended in Yssel's medium at 2×10⁶ cells/mL. 50 μL of cell suspension containing 80 U/mL IL-2 was added to each well of a flat bottom 96 well black plate. For the unstimulated control, IL-2 was omitted from the last column on the plate. Compounds were serially diluted in dimethyl sulfoxide (DMSO, 99.7% pure, cell culture tested, Sigma-Aldrich, St. Louis, Mo., Catalog No. D2650) from 5 mM in 3-fold dilutions, and then diluted 1:250 in Yssel's medium. 50 μL of 2× compound was added per well in duplicate and the cells were allowed to proliferate for 72 hours at 37° C.

Proliferation was measured using CellTiter-Glo® Luminescent Cell Viability Assay (Promega), which determines the number of viable cells in culture based on quantitation of the ATP present, as an indicator of metabolically active cells. The substrate was thawed and allowed to come to room temperature. After mixing the Cell Titer-Glo reagent and diluent together, 100 μL was added to each well. The plates were mixed on an orbital shaker for two minutes to induce lysis and incubated at room temperature for an additional ten minutes to allow the signal to equilibrate. Detection was performed using a Wallac Victor2 1420 multilabel counter purchased from Perkin Elmer, Shelton, Conn. The IC₅₀ calculated based on the results of this assay are provided in Table 1.

### Example 5

### A549 Epithelial Line Stimulated with IFNγ

The JAK activity of the compounds described herein may also be characterized by assaying the effect of compounds I and II described herein on A549 lung epithelial cells and U937 cells. A549 lung epithelial cells and U937 cells up-regulate ICAM-1 (CD54) surface expression in response to a variety of different stimuli. Therefore, using ICAM-1 expression as readout, test compound effects on different signaling pathways can be assessed in the same cell type. Stimulation with IL-1β through the IL-1β receptor activates the TRAF6/NFκB pathway resulting in up-regulation of ICAM-1. IFNγ induces ICAM-1 up-regulation through activation of the JAK1/JAK2 pathway. The up-regulation of ICAM-1 can be quantified by flow cytometry across a compound dose curve and EC₅₀ values are calculated. Exemplary assays of this type are described in greater detail below and in Example 6.

A549 lung epithelial cells up-regulate ICAM-1 (CD54) surface expression in response to a variety of different stimuli. Therefore, using ICAM-1 expression as readout, compound effects on different signaling pathways can be assessed in the same cell type. IFNγ up-regulates ICAM-1 through activation of the JAK/Stat pathway. In this example, the up-regulation of ICAM-1 by IFNγ was assessed.

The A549 lung epithelial carcinoma cell line originated from the American Type Culture Collection. Routine culturing was with F12K media (Mediatech Inc., Lenexa, Kans., Cat. No. 10-025-CV) with 10% fetal bovine serum, 100 I.U. penicillin and 100 ng/mL streptomycin (complete F12k media). Cells were incubated in a humidified atmosphere of 5% CO₂ at 37° C. Prior to use in the assay, A549 cells were washed with PBS and trypsinized (Mediatech Inc., Cat. No. 25-052-CI) to lift the cells. The trypsin cell suspension was neutralized with complete F12K media and centrifuged to pellet the cells. The cell pellet was resuspended in complete F12K media at a concentration of 2.0×10⁵/mL. Cells were seeded at 20,000 per well, 100 μL total volume, in a flat bottom tissue culture plate and allowed to adhere overnight.

On day two, A549 cells were pre-incubated with a 2,4-substituted pyrimidinediamine test compound or DMSO (control) (Sigma-Aldrich, St. Louis, Mo., Catalog No. D2650) for 1 hour. The cells were then stimulated with IFNγ (75 ng/mL) (Peprotech Inc., Rocky Hill, N.J., Cat. No. 300-02) and allowed to incubate for 24 hours. The final test compound dose range was 30 μM to 14 nM in 200 μA F12K media containing 5% FBS, 0.3% DMSO.

On day three, the cell media was removed and the cells were washed with 200 μL PBS (phosphate buffered saline). Each well was trypsinized to dissociate the cells, then neutralized by addition of 200 μL complete F12K media. Cells were pelleted and stained with an APC conjugated mouse anti-human ICAM-1 (CD54) (BD Pharmingen, San Diego, Calif., Catalog #559771) antibody for 20 minutes at 4° C. Cells were washed with ice cold FACS buffer (PBS+2% FBS) and surface ICAM-1 expression was analyzed by flow cytometry. Detection was performed using a BD LSR I System Flow Cytometer, purchased from BD Biosciences of San Jose, Calif. Events were gated for live scatter and the geometric mean was calculated (Becton-Dickinson CellQuest software version 3.3, Franklin Lakes, N.J.). Geometric means were plotted against the compound concentration to generate a dose response curve. The IC₅₀ calculated based on the results of this assay are provided in Table 1.

### Example 6

### U937 IFNγ ICAM1 FACS Assay

U937 human monocytic cells up-regulate ICAM-1 (CD54) surface expression in response to a variety of different stimuli. Therefore, using ICAM-1 expression as readout, compound effects on different signaling pathways can be assessed in the same cell type. IFNγ up-regulates ICAM-1 through activation of the JAK/Stat pathway. In this example, the up-regulation of ICAM-1 by IFNγ was assessed.

The U937 human monocytic cell line was obtained from ATCC of Rockville Md., catalog number CRL-1593.2, and cultured in RPM1-1640 medium containing 10% (v/v) FCS. U937 cells were grown in 10% RPMI. The cells were then plated at a concentration of 100,000 cells per 160 μL in 96 well flat bottom plates. The test compounds were then diluted as follows: 10 mM test compound was diluted 1:5 in DMSO (3 μL 10 mM test compound in 12 μL DMSO), followed by a 1:3 serial dilution of test compound in DMSO (6 μL test compound serially diluted into 12 μL DMSO to give 3-fold dilutions). Then 4 μL of test compound was transferred to 76 μL of 10% RPMI resulting in a 10× solution (100 μM test compound, 5% DMSO). For control wells, 4 μL of DMSO was diluted into 76 μL 10% RPMI.

The assay was performed in duplicate with 8 points (8 3-fold dilution concentrations from 10 μl) and with 4 wells of DMSO only (control wells) under stimulated conditions and 4 wells of DMSO only under unstimulated conditions.

The diluted compound plate was mixed 2× using a multimek (Beckman Coulter of Brea, Calif.) and then 20 μL of the diluted compounds was transferred to the 96 well plate containing 160 μL of cells, which were then mixed again twice at low speeds. The cells and compounds were then pre-incubated for 30 minutes at 37° C. with 5% CO₂.

The 10× stimulation mix was made by preparing a 100 ng/mL solution of human IFNγ in 10% RPMI. The cells and compound were then stimulated with 20 μL of IFNγ stimulation mix to give a final concentration of 10 ng/mL IFNγ, 10 μM test compound, and 0.5% DMSO. The cells were kept under conditions for stimulation for 18-24 hours at 37° C. with 5% CO₂.

The cells were transferred to a 96 well round bottom plate for staining and then kept on ice for the duration of the staining procedure. Cells were spun down at 1000 rpm for 5 minutes at 4° C., following which the supernatant was removed. Following removal of the supernatant, 1 μL APC conjugated mouse anti-human ICAM-1 antibody was added per 100 μL FACS buffer. The cells were then incubated on ice in the dark for 30 minutes. Following incubation, 150 μL of FACS buffer was added and the cells were centrifuged at 1000 rpm for 5 minutes at 4° C., following which the supernatant was removed. After removal of the supernatant, 200 μL of FACS buffer was added and the cells were resuspended. After suspension, the cells were centrifuged at 1000 rpm for 5 min at 4° C. Supernatant was then removed prior to resuspension of the cells in 150 μL FACS buffer.

Detection was performed using a BD LSR I System Flow Cytometer, purchased from BD Biosciences of San Jose, Calif. The live cells were gated for live scatter and the geometric mean of ICAM-APC was measured (Becton-Dickinson CellQuest software version 3.3, Franklin Lakes, N.J.). Both % live cells and ICAM-1 expression was analyzed. The assays for the test compounds were carried out in parallel with a control compound of known activity. The EC₅₀ for the control compound is typically 40-100 nM. The IC₅₀ calculated based on the results of this assay are provided in Table 1.

TABLE 1

| Compound | Example 3 | Example 4 | Example 5 | Example 6 |
|---|---|---|---|---|
| I | 0.056 | 0.181 | 11.338 | 0.565 |
| II | 9.655 | | | |
| III | 3.972 | | | |
| IV | 2.318 | 5.560 | | |
| V | 0.373 | | | 25.126 |
| VI | 0.104 | 0.262 | 4.973 | 0.424 |
| VII | 0.022 | 0.053 | | 0.140 |

### Example 7

### Pharmaceutical Formulations

This example describes pharmaceutical formulations containing compound I or II (which will be understood to also include salts thereof). Such formulations are prepared as known to those of skill in the art and additional formulations will be readily apparent to those of skill in the art upon consideration of this Example and additional disclosure herein.

| Formulation No. | Formulation Components |
|---|---|
| 1 | 50 mM pH 7.4 phosphate buffer, 0.05% Tween 80, 0.5% NaCl |
| 2 | 50 mM pH 7.4 phosphate buffer, 0.36% HPMC, 0.2% glycerin, 1% |
| | PEG400, 0.35% NaCl |
| 3 | 5 mM pH 7.4 phosphate buffer, 0.36% HPMC, 0.2% glycerin, 1% |
| | PEG400, 5% Cremophor ELP, 4.3% mannitol |
| 4 | 10 mM pH 5.8 citrate buffer, 4.2% mannitol |
| 5 | 10 mM pH 5.8 citrate buffer, 4.2% mannitol, 0.36% HPMC, 0.2% |
| | glycerin |
| 6 | 0.3% tyloxapol, 0.5% Carbopol974P, 2.25% mannitol, 50 mM pH 6.5 |
| | phosphate buffer, 230 mOsm/kg |
| 7 | 0.3% tyloxapol, 0.1% Carbopol974P, 2.25% mannitol, 50 mM pH 6.5 |
| | phosphate buffer, 230 mOsm/kg |

Each of the above formulations, 1-7, are prepared with compound I or II in three dosage concentrations: 0.001%, 0.003% and 0.01% (w/w). Each formulation is prepared by adding the specified amount of a tonicity agent (mannitol) to a flask, heating to about 50° C. in about half the final volume of the specified buffer (phosphate or citrate). After heating, the appropriate amount of compound I or II is added along with the additional excipients (glycerin and/or PEG400) as indicated. Purified water is added in sufficient quantity. The mixture is stirred to homogeneity (about five minutes) and then filtered through a sterilizing filter membrane into a sterile vessel. If necessary, pH is adjusted by addition of 1.0 N NaOH.

Optionally, formulations having a higher concentration of compound I or II (for example, 0.03% w/w) can include a surfactant and optionally a stabilizing polymer. With reference to formulations 6 and 7, preferred surfactants include Triton X114 and tyloxapol, which are commercially available from Sigma-Aldrich (of St. Louis, Mo.) and Pressure Chemical Company (of Pittsburgh, Pa.), respectively. Preferred stabilizing polymers include the carbomer Carbopol 974p (commercially available from Lubrizol, of Wickliffe, Ohio).

Formulations 6 and 7 are prepared by dispersing the carbomer first in the surfactant containing buffer at 10× of their final concentration (e.g. 3% tyloxapol in 50 mM phosphate buffer at pH 6.5 with 2.5% mannitol and 5% Carbomer 9′74p). Either compound I or compound II is then dispersed in this preconcentrate also at 10× of its final concentration. The mixture is homogenized, with final formulation being obtained by 10× dilution of filtered preconcentrate in a matching buffer.

### Example 8

### Induced Dry Eye Mouse Model

This example describes the treatment of symptoms in a mouse model of dry eye. An injection was prepared comprising 2.5 mg/mL scopolamine (Sigma-Aldrich) in injectable saline (1-1.5 mL per animal). Normal C57 mice are injected with 200-250 μL of scopolamine solution four times every 2.5 hours in alternating hindquarters. The mice are placed in special cages (with holes in front and back) and placed in a hood. Fans are placed in front of each cage and run for 16 hours overnight for five consecutive days. Measurements of tear production are taken daily and at the end of five days all mice are considered dry-induced. Animals are treated with drug or vehicle (one of formulations 1-7 described above) at 1 μL, once per day for two weeks. Tear production is measured with treatment results being measured by restoration in part or in whole of normal tear production values.

Tear production can be measured either by quantitative means, or qualitative assessment of the animal's corneas can be performed, for example by biomicroscopic examination of fluorescein or rose bengal staining patterns of the treated eyes. A reduction in such staining is indicative of successful treatment of the dry eye condition.

### Example 9

### Other Animal Models

Other animal models of dry eye can be used to demonstrate the activity of the disclosed agents in treating this condition. For example, several models of Sjogren's syndrome have been developed that mimic the early activation and infiltration of autoreactive lymphocytes seen in that condition. The nonobese diabetic (NOD) mouse model shows a lymphocytic infiltration of predominantly CD4⁺ Th1 cells in the lacrimal gland as well as other organs, including the pancreas, submandibular, and thyroid glands. Male NOD mice show significant inflammatory lesions of the lacrimal gland from the age of 8 weeks, whereas female NOD mice do not show any changes until 30 weeks of age. Takahashi et al., High incidence of autoimmune dacryoadenitis in male non-obese diabetic (NOD) mice depending on sex steroid. Clin Exp Immunol. 1997; 109:555-561. The MRL/MpJ-fas⁺/fas⁺ (MRL/+) and MRL/MpJ-fas^{lpr}/fas^{lpr} (MRL/lpr) mouse models of Sjögren's syndrome exhibit lacrimal gland infiltrates characterized by a predominance of CD4⁺ T cells. Van Blokland and Versnel, Pathogenesis of Sjögren's syndrome: characteristics of different mouse models for autoimmune exocrinopathy. Clin Immunol. 2002; 103:111-124.

Because of the large exposed ocular surface in rabbits compared with mice, standard dry eye clinical tests such as tear break-up time and fluorescein or rose bengal staining of the ocular surface can be much more easily performed in rabbits. An autoimmune disease in rabbits resembling Sjögren's syndrome can be provoked by injecting into the lacrimal gland autologous peripheral blood lymphocytes proliferated in culture with epithelial cells obtained from the contralateral excised gland.

These and other animal models are used to test the claimed compounds, as well as combination formulations, such as those described herein. The formulations are administered to the animal, and the eyes are examined for evidence of increased tear production or decreased evidence of dry eyes.

### Example 10

### Methods of Treatment and Combination Formulations

Subjects to be treated with the claimed formulations are selected based on a clinical presentation or ophthalmic examination that suggests the presence of dry eyes. For example, the subject may complain of an uncomfortable or burning sensation of the eyes. Photophobia or blurred vision may even be present in severe cases. The medical history of the patient may also be suggestive of dry eyes, for example in a patient with a pre-existing diagnosis of acne rosacea, radiation therapy, rheumatoid arthritis, systemic lupus erythematosus, or scleroderma, or other autoimmune disorder. Biomicroscopic examination with a slit lamp is typically performed to detect meibomitis, conjunctival dilation, decreased tear meniscus, increased tear debris, mucus strands or staining patterns consistent with keratoconjunctivitis sicca. A tear breakup time of less than 10 seconds may also be assessed, and the Schirmer test is frequently performed to more objectively identify subjects who would benefit from treatment with the claimed agents.

Particular groups of patients may be selected for treatment, for example those who have decreased tear production from the lacrimal glands (for example, those who have a Schirmer's test that suggests hypofunction of the lacrimal gland due to immune-mediated or other disorders). The claimed compositions are instilled in the eye using eye drops or ointments, two to four times a day. Treatment may be continued for at least a week, month, or year, and in some subjects treatment may extend over multiple years.

In particular cases, subjects are selected for comcomitant treatment with other pharmaceutical or non-pharmaceutical interventions. For example, punctal occlusion is performed to decrease the outflow of tears from the eye while the claimed composition increases lacrimal gland tear production.

Combination therapies are also provided that combine the compounds of formula I and/or II (which includes salts thereof) with another agent that treats another condition, such as a condition associated with the dry eyes. In some examples, the subject is diagnosed with an underlying disorder associated with the dry eyes and the combination therapy is administered to the subject. In one example, the subject is found to have a meibomitis that would be responsive to topical application of corticosteroids, such as a prednisolone acetate ophthalmic syspension 1%. The compounds of formula I and/or II (which includes salts thereof) are suspended in the prednisolone formulation and instilled in the eye 2 to 4 times a day. In other examples, the dry eyes are associated with seasonal allergies or other inflammatory conditions, and the eye drops are administered with or in a formulation that includes antihistamines (such as pheniramine, emedastine, or azelastine), decongestants (such as tetrahydrozoline hydrochloride or naphazoline), or a non-steroidal anti-inflammatory agent (such as nepafenac or ketorolac), corticosteroids (such as fluorometholone or loteprednol), mast cell stabilizers (such as azelastie, cromal, emedastine, ketotifen, Iodoxamine, nedocromil, olopatadine, or pemirolast). If the dry eyes are associated with an infectious bacterial condition (such a meibomian gland infection or corneal infection) the eye drops are administered with or in a combination formulation can contain appropriate antibiotics (such as ciprofloxacin, erythromycin, gentamicin, ofloxacin, sulfacetamine, tobramycin, or monofloxacin). If the dry eyes are associated with a viral infection, the eye drops are administered with or in a combination formulation with an anti-viral agent such as trifluridine or idoxuridine.

Another example of a combination therapy is a subject who is diagnosed with ocular rosacea after presenting with irritated eyes and facial erythema with telangiectasia. The subject is treated with eye drops that contain the compounds of formula I and/or II, and the subject is also treated with an oral antibiotic, such as a tetracycline antibiotic, such as minocycline.

In another example, the subject presents with dry eyes and another pre-existing autoimmune disorder, and is treated with the eye drops that contain the compounds of formula I and/or II. The subject is also treated with systemic (for example) oral corticosteroid therapy, such as a tapering dose of prednisolone.

We claim:

1. A method of treating a disease and/or disorder of the eye, comprising administering to a subject an effective amount of a compound of formula I and/or II, or a pharmaceutically acceptable salt form thereof.

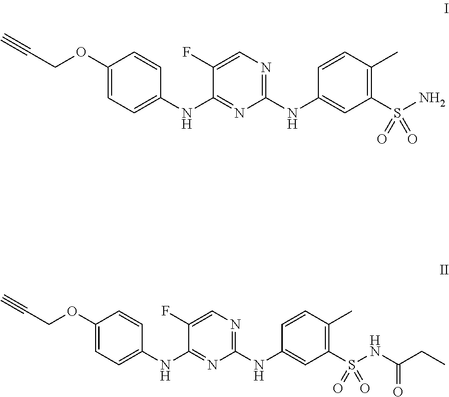

2. The method of claim 1, wherein the disease and/or disorder of the eye is selected from dry eye syndrome, diabetic retinopathy, macular degeneration uveitis, allergic conjunctivitus, glaucoma, rosacea and combinations thereof.

3. The method of claim 1, wherein the disease and/or disorder of the eye is dry eye syndrome.

4. The method of claim 1, wherein the disease and/or disorder of the eye is uveitis.

5. The method of claim 1, wherein the disease and/or disorder of the eye is allergic conjunctivitus.

6. The method of claim 1, wherein the disease and/or disorder of the eye is glaucoma.

7. The method of claim 1, wherein the disease and/or disorder of the eye is rosacea.

8. The method of claim 1, wherein the pharmaceutically acceptable salt form thereof is a salt of compound II.

9. The method of claim 8, wherein the salt of compound II is selected from the sodium salt, the potassium salt, the calcium salt, the arginine salt and the choline salt.

10. The method of claim 1, wherein the compound of formula I and/or II, or the pharmaceutically acceptable salt form thereof, is administered either in combination or adjunctively with an anti-inflammatory, an antihistamine, an antibiotic, an antiviral and a glaucoma medication.

11. A pharmaceutical formulation comprising compound I and/or compound II, where the formulation is selected from a solution, a gel, an ointment, a cream and a suspension.

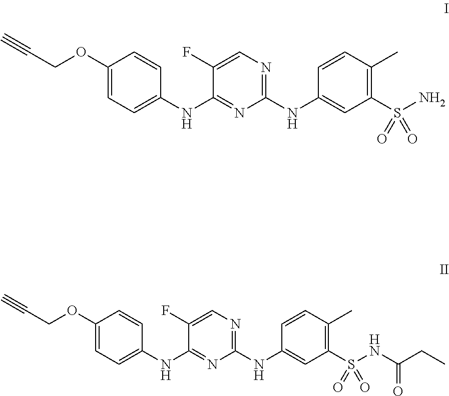

12. A kit comprising a pharmaceutical formulation comprising compound I and/or compound II, or a pharmaceutically acceptable salt form thereof, for administration of the pharmaceutical formulation to the eye. [chemical structure]
